(19) United States
(12) Patent Application Publication
(10) Pub. No.: US 2010/0134762 A1
(43) Pub. Date: Jun. 3, 2010

(54) ADJUSTABLE EYEGLASS FRAME

(76) Inventor: Shing Chuan Liu, Taichung (TW)

Correspondence Address: SAM CHEN, 7F-1, 293, ROOSEVELT ROAD, SEC 3, TAIPEI, TW

(21) Appl. No.: 12/324,958

(22) Filed: Nov. 28, 2008

(51) Int. Cl. A61B 3/04

(52) U.S. Cl. 351/230

(57) ABSTRACT

An eyeglass frame includes left and right length adjustable temples; left and right viewing assemblies adjustably mounted in left and right frames respectively; and a length adjustable bridge. Either viewing assembly includes a transversely moveable frame element slidably secured to the left or right frame; a longitudinally moveable frame element slidably secured to the transversely moveable frame element; a front aperture member slidable about the longitudinally moveable frame element and including an upper longitudinal slot and a cut on an inner side; a corresponding rear aperture member slidable about the longitudinally moveable frame element and including a lower longitudinal slot and a cut on an inner side; and a circular member rotatably secured to the longitudinally moveable frame element and including two projections moveably disposed in the slots respectively. An aperture is formed by at least the cuts, whereby rotating the circular member will change size of the aperture.

### BACKGROUND OF THE INVENTION

1. Field of Invention

The invention relates to eyeglass frame and more particularly to the frame of a pair of eyeglasses capable of adjusting its size in order to correctly determine a prescription for child eyeglasses as the child grows.

2. Description of Related Art

Myopia may occur before 20 years of old, particularly the school-age years. It is typically for a child to wear a pair of eyeglasses equipped with one or two corrective lenses when he or she begins to have the symptom of myopia. However, this type of conventional corrective lenses has a couple of drawbacks as detailed below.

There is only one aperture on the lens and thus it is impossible of adjusting its diameter if such need arises. It is typical to prepare a number of eyeglasses each having two lenses each with an aperture being different from the others in diameter. Hence, a wearer may choose a pair of eyeglasses with correct apertures from these eyeglasses. This is very inconvenient.

The frame of the typical eyeglasses is fixed in size. It is understood that a child may grow rapidly in a short period of time during the school-age years. Hence, for example, a child may not be able to wear a pair of eyeglasses bought several months ago because his or her head has grown too large to wear it. The only solution is to discard it and buy a pair of new eyeglasses. This is not economical. Thus, the need for improvement still exists.

### SUMMARY OF THE INVENTION

It is therefore one object of the invention to provide a frame of a pair of eyeglasses capable of adjusting its size in order to correctly determine a prescription for child eyeglasses as the child grows.

The above and other objects, features and advantages of the invention will become apparent from the following detailed description taken with the accompanying drawings.

### BRIEF DESCRIPTION OF THE DRAWINGS

FIG. 1 is a perspective view of a preferred embodiment of eyeglasses according to the invention;

FIG. 2 is an exploded perspective view of the left portion of FIG. 1;

FIG. 3 is an enlarged view of the components except the frame and the temple of FIG. 2;

FIG. 4 is a longitudinal sectional view of the eyeglasses of FIG. 1;

FIG. 5 is a longitudinal sectional view of the assembled viewing assembly of FIG. 3;

FIG. 6 is a front view of the assembled viewing assembly of FIG. 3;

FIG. 7 is an exploded view of the bridge of the eyeglasses;

FIG. 8 is a longitudinal sectional view of the assembled bridge of FIG. 8;

FIG. 9 is a transverse sectional view of the assembled bridge of FIG. 8; and

FIG. 10 is an exploded view of the left temple of FIG. 1.

### DETAILED DESCRIPTION OF THE INVENTION

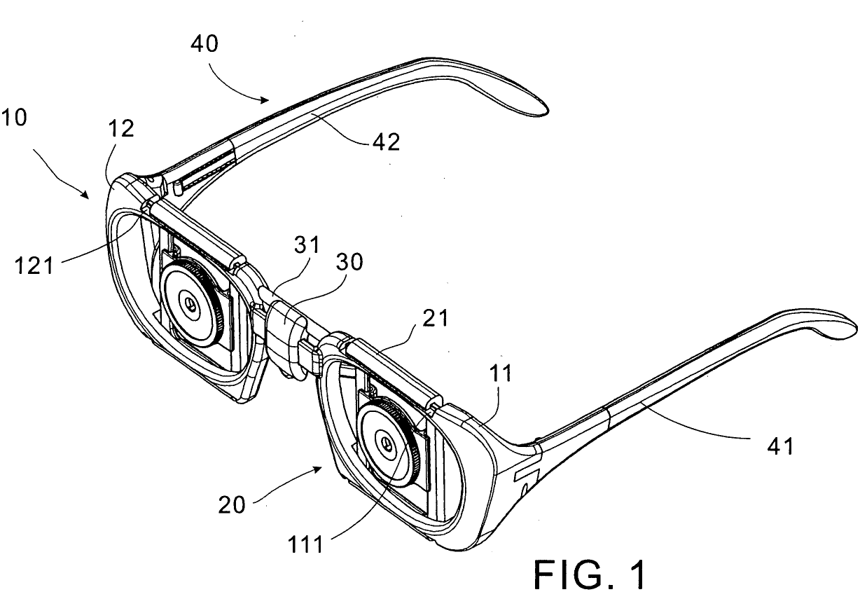

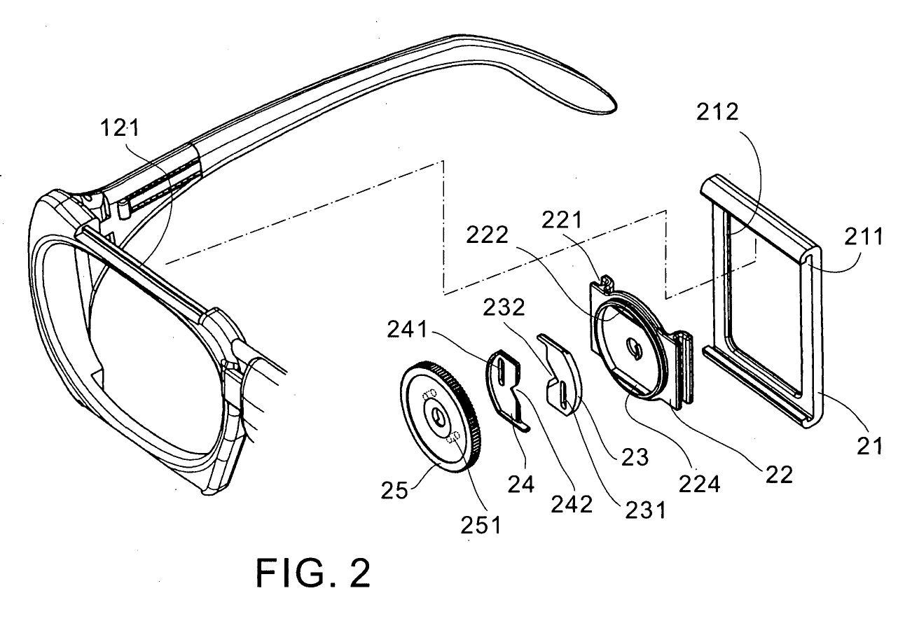

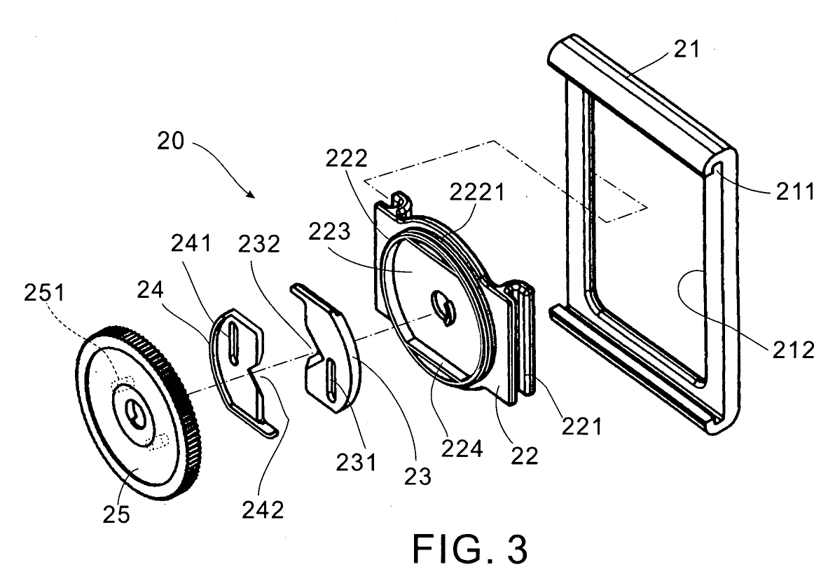

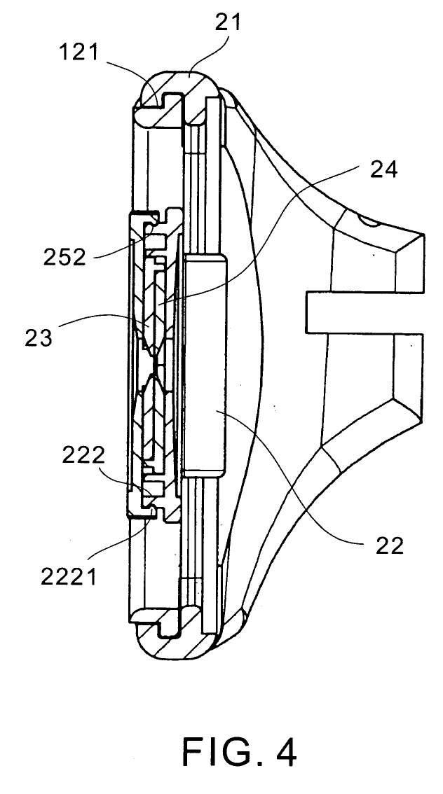

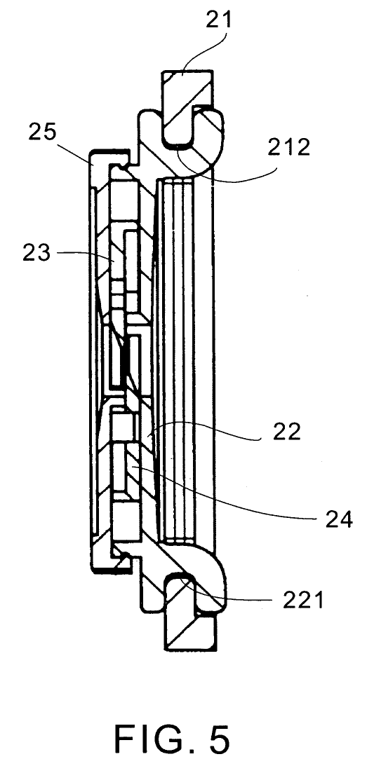

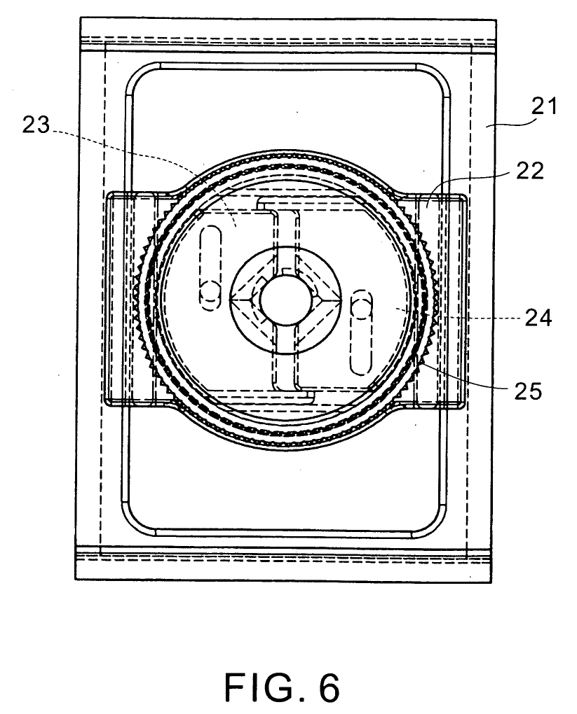

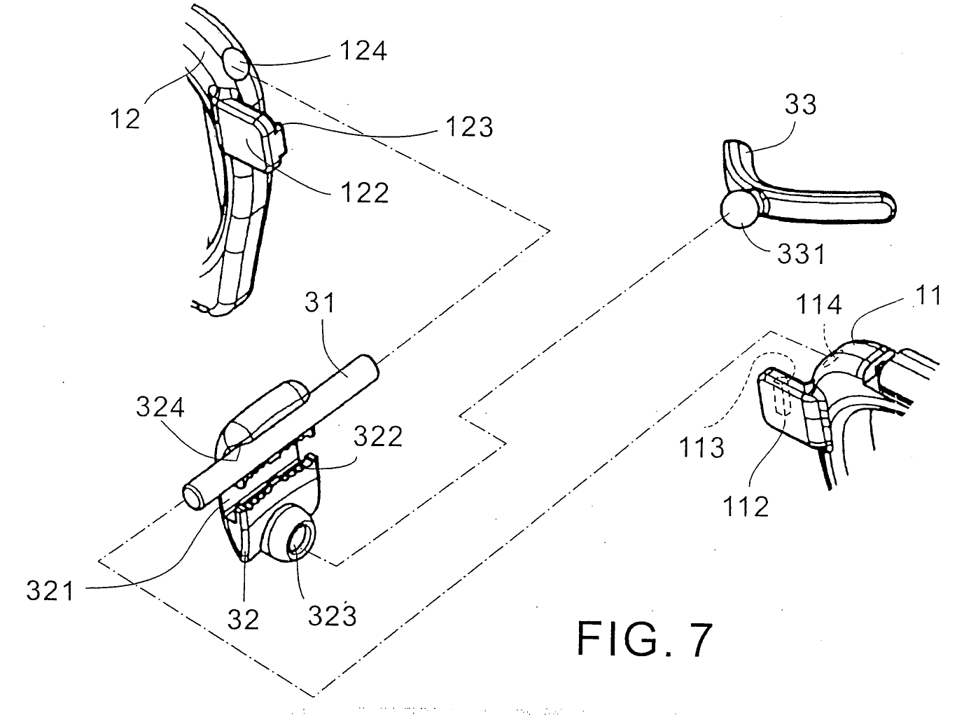

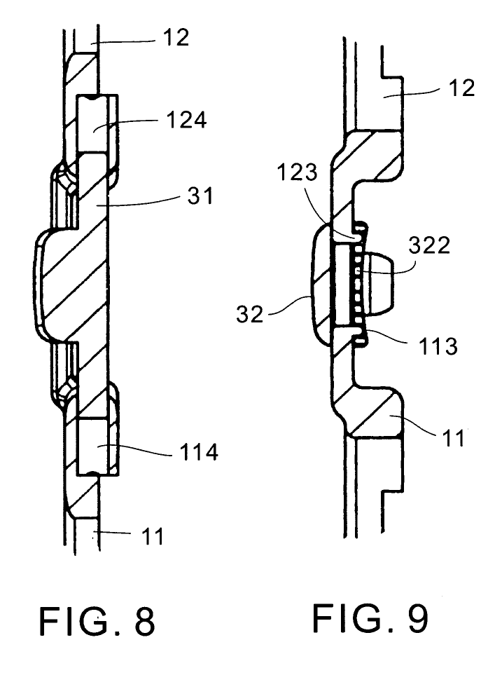

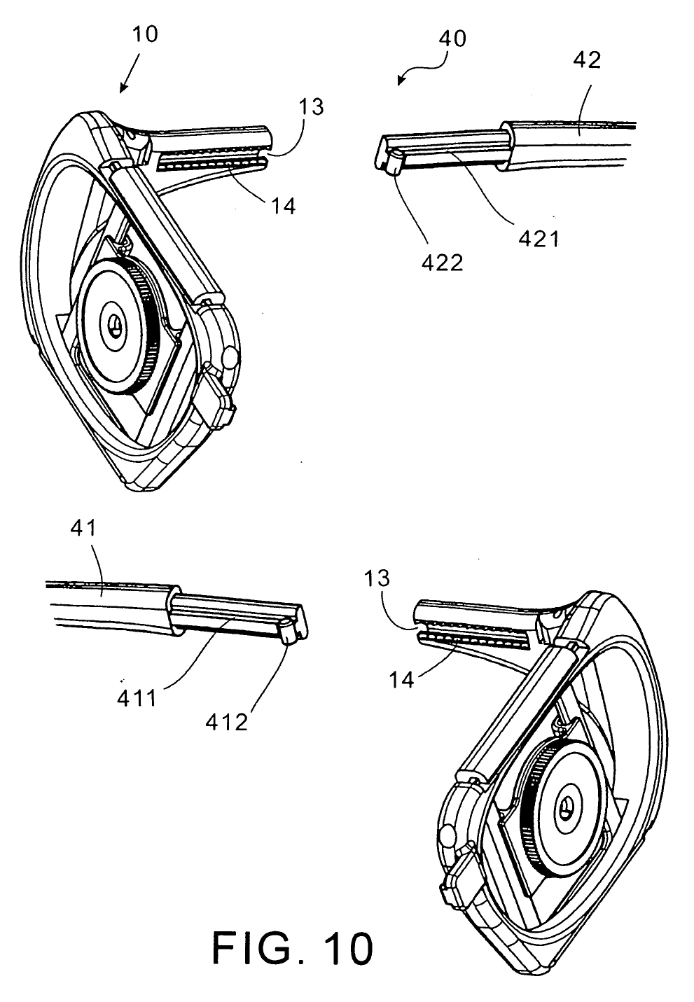

Referring to FIGS. 1 to 10, a pair of eyeglasses 10 in accordance with a preferred embodiment of the invention is shown. The eyeglasses 10 comprises the following components each being discussed in detail below.

A frame comprises a left frame 11 and a right frame 12 each equipped with a viewing assembly 20. A bridge 30 is provided to interconnect the left frame 11 and the right frame 12. A temple assembly 40 comprises a left temple 41 and a right temple 42. The left frame 11 has upper and lower rails 111 on the top and bottom edges respectively, a transverse hole 114 proximate an inner end of the upper rail 111, a transverse protrusion 112 slightly below the transverse hole 114 facing the right frame 12, and a pair of opposite tabs 113 extending rearward from the open end of the protrusion 112. Likewise, the right frame 12 has upper and lower rails 121 on the top and bottom edges respectively, a transverse hole 124 proximate an inner end of the upper rail 121, a transverse protrusion 122 slightly below the transverse hole 124 facing the left frame 11, and a pair of opposite tabs 123 extending rearward from the open end of the protrusion 122.

For example, the right viewing assembly 20, as a mirror image of the left viewing assembly 20, comprises a transversely moveable frame element 21 of hollow rectangle, the transversely moveable frame element 21 having upper and lower grooves 211 slidably secured to the upper and lower rails 121 by snapping respectively, and left and right rails 212.

The right viewing assembly 20 further comprises a longitudinally moveable frame element 22 of substantially rectangular, the longitudinally moveable frame element 22 including left and right troughs 221 slidably secured to the left and right rails 212 respectively, an annular flange 222 on one surface distal the transversely moveable frame element 21, an annular cavity 2221 adjacent to the outer portion of the flange 222 and being concentric therewith, a space 223 confined by the flange 222, a central hole (not numbered) through the bottom of the space 223, and upper and lower transverse ribs 224 in the space 223, each rib 224 having both ends fixedly secured to two opposite points of the flange 222.

The right viewing assembly 20 further comprises a front aperture member 23 and a corresponding rear aperture member 24. The front aperture member 23 comprises an upper longitudinal slot 231, a triangular cut 232 on the center of an inner side, and an outer side being arcuate in shape. Likewise, the corresponding rear aperture member 24 comprises a lower longitudinal slot 241, a triangular cut 242 on the center of an inner side, and an outer side being arcuate in shape. The top and bottom edges of the front aperture member 23 are adapted to slide about the upper and lower transverse ribs 224 and the top and bottom edges of the corresponding rear aperture member 24 are adapted to slide about the upper and lower transverse ribs 224 respectively.

The right viewing assembly 20 further comprises a circular toothed member 25 including a central through hole (not numbered), a peripheral ridge 252 projecting inward from the teeth and facing the transversely moveable frame element 21, and two spaced projections 251 on one surface facing the transversely moveable frame element 21. The projections 251 are disposed in the slots 231, 241 respectively and are adapted to slide in a distance defined by the length of each of the slots 231, 241. The ridge 252 is matingly rotatably secured to the cavity 2221 for mounting the circular toothed member 25 onto the longitudinally moveable frame element 22 with the front aperture member 23 and the corresponding rear aperture member 24 disposed therebetween. A complete aperture is formed by the central through hole of the circular toothed member 25, the two cuts 232, 242 (the main factors), and the central through hole of the longitudinally moveable frame element 22. Thus, a rotation of the circular toothed member 25 by rotating the teeth thereof may transversely move both the front aperture member 23 and the corresponding rear aperture member 24 so as to change a position of the front aperture member 23 relative to the corresponding rear aperture member 24 (i.e., the front aperture member 23 may partially cover the corresponding rear aperture member 24) and thus the size of the aperture.

The bridge 30 comprises an upper transverse channel 324 of arcuate section, a bar 31 of circular section having its intermediate portion fastened in the channel 324, an intermediate transverse tunnel 321, two toothed elements 322 on the upper and lower edges of the tunnel 321 respectively, and a lower well 323 of substantially half-spherical. All of the above components of the bridge 30 are provided on its rear surface. Both ends of the bar 31 are slidably disposed in the holes 114, 124 respectively. The tabs 113 are frictionally slidably secured to the left portions of the toothed elements 322 and the tabs 123 are frictionally slidably secured to the right portions of the toothed elements 32 respectively. The bridge 30 further comprises an arcuate pad 33 having a front spherical member 331 rotatably fastened in the well 323.

A channel 13 of substantially T-section is provided on an inner surface of a pivotal member (not numbered) projecting rearward from the end of the left frame 11 or the right frame 12. The channel 13 has an open rear end. Upper and lower teeth 14 are provided on upper and lower edges of the channel 13 respectively. The right temple 42 has a forward rail 421 of substantially T-section, and a short longitudinal cylinder 422 at the open end of the rail 421. Likewise, the left temple 41 has a forward rail 411 of substantially T-section, and a short longitudinal cylinder 412 at the open end of the rail 411. The rail 421 or 411 is frictionally slidably disposed in the channel 13. For example, a user may push or pull the cylinder 422 to adjust the length of the right temple 42.

Moreover, adjustment of a distance between the left frame 11 and the right frame 12 can be done by, for example, pushing or pulling the protrusion 112 of the left frame 11 along the tunnel 321 with the tabs 113 frictionally moving along the teeth of the toothed elements 322 and the both ends of the bar 31 slidably moving with respect to the holes 114, 124 respectively.

Further, a person, e.g., a physician, after aligning the aperture with the pupil of a child may further adjust the size of the aperture by rotating the circular toothed member 25 by moving the front aperture member 23 to partially cover the corresponding rear aperture member 24 or not. The longitudinal position of the longitudinally moveable frame element 22 relative to the transversely moveable frame element 21 can be adjusted by sliding the troughs 221 along the rails 212. The transverse position of the transversely moveable frame element 21 relative to, for example, the left frame 11 can be adjusted by sliding the grooves 211 along the rails 121.

While the invention herein disclosed has been described by means of specific embodiments, numerous modifications and variations could be made thereto by those skilled in the art without departing from the scope and spirit of the invention set forth in the claims.

What is claimed is:

1. An eyeglass frame comprising:
left and right length adjustable temples;
left and right frames;
left and right viewing assemblies adjustably mounted in the left and right frames respectively; and
a length adjustable bridge interconnecting the left and right fames,
wherein each of the left and right frames comprises an upper transverse hole open to the bridge, a transverse protrusion extending toward the bridge, and a pair of opposite tabs extending rearward from the open end of the transverse protrusion;
wherein the bridge comprises an upper bar having both ends slidably disposed in the upper transverse holes of the left and right frames respectively, an intermediate transverse tunnel having upper and lower toothed edges with the opposite tabs frictionally slidably secured to the upper and lower toothed edges of the intermediate transverse tunnel respectively, and a lower rotatable pad;
wherein each of the left and right viewing assemblies comprises a transversely moveable frame element slidably secured to the left or right frame; a longitudinally moveable frame element slidably secured to the transversely moveable frame element and including an annular flange on one surface distal the transversely moveable frame element, an annular cavity adjacent to the outer portion of the annular flange, a central through hole, and upper and lower transverse ribs in a space defined by the annular flange; a front aperture member slidable about the upper and lower transverse ribs and including an upper longitudinal slot and a triangular cut on the center of an inner side; a corresponding rear aperture member slidable about the upper and lower transverse ribs and including a lower longitudinal slot and a triangular cut on the center of an inner side; and a circular member including peripheral teeth, a central through hole, two projections moveably disposed in the longitudinal slots respectively, and a peripheral ridge projecting inward from the peripheral teeth to rotatably secure to the annular cavity and mount the front aperture member and the corresponding rear aperture member between the circular member and the longitudinally moveable frame element;
wherein an aperture is formed by the central through hole of the circular member, the triangular cuts, and the central through hole of the longitudinally moveable frame element; and
wherein the circular member is adapted to rotate to transversely move both the front aperture member and the corresponding rear aperture member so as to change both a position of the front aperture member relative to the corresponding rear aperture member and the size of the aperture.

2. The eyeglass frame of claim 1, wherein each of the left and right length adjustable temples comprises a pivotal member projecting rearward a predetermined distance from its end, the pivotal member having a channel on its inner surface and upper and lower teeth on upper and lower edges of the channel respectively, and a rear member having a forward rail frictionally slidably disposed in the channel, and a trigger at the open end of the rail.
